SEQUENCE LISTING (1 sequence)

SEQ ID NO: 1 - protein, 30 aa, synthetic construct
RGDRGDMHSHRDFQPVLHLVALNSPLSGGM

(19) United States
(12) Patent Application Publication
(10) Pub. No.: US 2024/0207367 A1
(43) Pub. Date: Jun. 27, 2024

(54) POLYPEPTIDE DRUG FOR PREVENTING AND/OR TREATING NEUROBLASTOMA AND USE THEREOF

(71) Applicant: ZHUHAI TENGBAI PHARMACEUTICAL CO., LTD., Zhuhai (CN)

(72) Inventors: Xinchun YANG, Zhuhai (CN); Jinlong ZHAO, Zhuhai (CN); Baofeng YANG, Zhuhai (CN); Ye YUAN, Zhuhai (CN); Yu LIU, Zhuhai (CN); Zhun WANG, Zhuhai (CN)

(21) Appl. No.: 18/598,527

(22) Filed: Mar. 7, 2024

Related U.S. Application Data
(63) Continuation of application No. 17/799,552, filed on Aug. 12, 2022, now abandoned.

(30) Foreign Application Priority Data
Feb. 13, 2020 (CN) 202010089778.X

(51) Int. Cl. A61K 38/39; A61K 47/26; A61P 25/00; A61P 35/00

(52) U.S. Cl. CPC A61K 38/39; A61K 47/26; A61P 25/00; A61P 35/00

(57) ABSTRACT

A polypeptide, a composition containing the polypeptide, and a use thereof in preparing a drug for preventing and/or treating neuroblastoma.

### CROSS-REFERENCE TO RELATED APPLICATIONS

This application is a continuation of U.S. patent application Ser. No. 17/799,552, filed on 12 Aug. 2022, which is a U.S. National Stage application of International Patent Application No. PCT/CN2020/075599, filed on 17 Feb. 2020, which claims the benefit of Chinese Patent Application No. 202010089778.X, filed on 13 Feb. 2020, the entire disclosures of which are hereby incorporated by reference in their entireties.

### SEQUENCE LISTING

The instant application contains a Sequence Listing which has been submitted electronically in XML format and is hereby incorporated by reference in its entirety. Said XML copy, created on 6 Mar. 2024, is named 0361_0015_CON_ST25.xml and is 1,780 bytes in size.

### TECHNICAL FIELD

The present invention relates to the field of pharmaceutical biology, and in particular to a use of a polypeptide in the preparation of medicament for preventing and/or treating neuroblastoma and a composition comprising the polypeptide.

### BACKGROUND

Neuroblastoma (NB) is the most common extracranial solid tumor in children under 5 years old, accounting for 6% to 10% of childhood tumors and 15% of childhood tumor mortality, which is the most common malignant tumor in children under 1 year old. Neuroblastoma is an embryonal tumor of neural crest origin, consisting of undifferentiated sympathetic nerve cells. Neuroblastoma has an insidious onset and is not easy to diagnose at an early stage. About 70% of children with neuroblastoma have metastases by the time of consultation, wherein common metastatic sites include kidney and bone marrow. The tumor is highly malignant, progresses rapidly, prone to metastasis and life-threatening, and the effect of surgical treatment therefor is very limited. Traditional chemotherapy regimens are the first-line regimens for high-risk neuroblastoma. The overall survival rate of neuroblastoma patients has been greatly increased due to the updated and improved treatment methods, but the 5-year survival rate is still less than 40%, and there is no significant improvement for the survival rate and quality of life. Neuroblastoma is a type of embryonal tumor, similar to other solid tumors, which means that most patients are sensitive to chemotherapy. In recent years, a treatment regimen has been become a widely recognized principle of treatment that preoperative neoadjuvant chemotherapy is performed for patients with neuroblastoma who cannot undergo surgical resection of the tumor, followed by postponement or secondary surgery after the tumor has shrunk in size. Comprehensive treatment such as chemotherapy, radiotherapy and immunotherapy is performed additionally after the surgery, such treatment can obtain a better outcome. Commonly used chemotherapy medicament include: Cyclophosphamide (CTX), Ifosfamide (IFO), Vincristine (VCR), Cisplatin (DDP), Etoposide, Teniposide and Adriamycin (ADR). Chemotherapy medicament mainly kill cells that are actively dividing, however, the actively dividing cells include hair follicle cells and blood cells in addition to cancer cells, which leads to hair loss and bone marrow suppression after chemotherapy, further resulting in hair loss and requirement for bone marrow transplantation after chemotherapy. Besides, it is easy to cause toxic side effects such as infection. At the same time, the therapeutic effect of chemotherapeutic medicament on neuroblastoma and the tolerance of patients are already close to the maximum and prone to medicament resistance.

Therefore, it is still very urgent and necessary to find effective substances or medicament for the treatment of neuroblastoma.

### CONTENT OF THE PRESENT INVENTION

The inventor of the present application found in their research that a polypeptide HYD-PEP06 showed an inhibitory effect on neuroblastoma, wherein the polypeptide has an amino acids sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

Therefore, the first aspect of the present invention relates to a use of a polypeptide in the preparation of a medicament for treating neuroblastoma, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

The second aspect of the present invention relates to a polypeptide for use in treating neuroblastoma, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

The third aspect of the present invention relates to a method for treating neuroblastoma, wherein the method comprises administering to a subject in need thereof a therapeutically effective amount of a polypeptide, the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

The fourth aspect of the present invention relates to a composition comprising a polypeptide and an adjuvant, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1), the composition is used for preventing and/or treating of neuroblastoma.

### DETAILED DESCRIPTION OF THE INVENTION

The term “HYD-PEP06” as used herein refers to a polypeptide having an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (see SEQ ID No. 1). The Chinese patent application No. 201410658306.6 is incorporated herein by reference in its entirety.

The term “prevention” as used herein refers to the ability of the composition or medicament to reduce the frequency or delay the onset of symptoms of a medical condition in a subject compared to a subject not administered the composition or medicament when used for a disease or condition (e.g., a tumor).

The term “treatment” as used herein refers to alleviating, relieving or improving the symptoms of a disease or condition (e.g., a tumor), improving the underlying metabolically induced symptoms, or suppressing a disease or condition, such as stopping the progression of a disease or condition, relieving a disease or condition, causing a disease or condition to subside, relieving a condition caused by a disease or condition, or stopping symptoms of a disease or condition.

The term “metastatic neuroblastoma” as used herein includes neuroblastoma resulting from metastasis of tumors other than neuroblastoma, as well as tumor resulting from metastasis of neuroblastoma to other organs and/or tissues of the body (e.g., Tumors from bone, bone marrow, lymph nodes, liver, skin, lung (lung metastases are a marker of poor prognosis), and brain parenchyma). Tumors resulting from metastasis of neuroblastoma to other organs and/or tissues of the body include, but are not limited to: liver metastases from neuroblastoma, lung metastases from neuroblastoma, brain metastases from neuroblastoma, bone metastases from neuroblastoma, lymphoid metastases from neuroblastoma, and myeloid metastases from neuroblastoma.

The term “tumor” as used herein refers to new organisms formed by the proliferation of local tissue cells under the action of various tumorigenic factors. Because these new organisms are mostly space-occupying massive protrusions, such new organisms are also called neoplasms. Tumors can be classified as benign or malignant. Malignant tumors can also be classified as cancers or sarcomas.

The term “subject” as used herein refers to include humans (e.g., human patients) and animals (e.g., mice, rats, dogs, cats, rabbits, chickens, or monkeys, etc.). When the subject is a human patient (the body weight is usually calculated as 60 kg), unless otherwise stated, the dosage described in the present invention can be converted using a conversion factor of experimental animals (e.g., human dosage=mouse dosage/12.3) (see Kin Tam. “Estimating the “First in human” dose-a revisit with particular emphasis on oncology medicament, ADMET & DMPK 1(4) (2013) 63-75). Those skilled in the art can reasonably adjust the dosage according to the common knowledge, the specific body weight of the subject, the type and severity of the disease and other factors, and the technical solutions with these adjustments all fall into the protection scope of technical solution claimed by the present invention.

The term “room temperature” as used herein refers to 25° C.±1° C. Besides, experimental temperatures without detailed description are room temperature.

The term “about” as used herein refers to ±10%, more preferably ±5%, and most preferably ±2% of the numerical value that the term modifies, so that those skilled in the art can clearly determine the value of the modified numerical value.

The first aspect of the present invention relates to a use of a polypeptide in the preparation of a medicament for preventing and/or treating neuroblastoma, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

In some embodiments, wherein the medicament is in a form of a white or off-white loose cake or powder. Further, the medicament should be diluted with 5% glucose solution (e.g., glucose injection), not with sodium chloride injection.

In some embodiments, wherein the medicament can be administered by intravenous injection.

In some embodiments, wherein the neuroblastoma is neuroblastoma in situ or metastatic neuroblastoma. Further, the neuroblastoma is liver metastases from neuroblastoma, lung metastases from neuroblastoma, brain metastases from neuroblastoma, bone metastases from neuroblastoma, lymphatic metastases from neuroblastoma, and myeloid metastases from neuroblastoma.

In some embodiments, the medicament is in a form of unit dosage. Further, the unit dosage comprises the following amounts of the polypeptide: about 0.6 mg, about 3 mg, about 6 mg, about 15 mg, about 24 mg, about 48 mg, about 96 mg, about 120 mg, about 150 mg, about 300 mg, about 600 mg, or a range between foregoing amounts of the polypeptide, for example, 0.6 mg-600 mg, 0.6 mg-300 mg, 0.6 mg-150 mg, 0.6 mg-120 mg, 0.6 mg-96 mg, 0.6 mg-48 mg, 0.6 mg-24 mg, 0.6 mg-15 mg, 0.6 mg-6 mg, 0.6 mg-3 mg, 3 mg-600 mg, 3 mg-300 mg, 3 mg-150 mg, 3 mg-120 mg, 3 mg-96 mg, 3 mg-48 mg, 3 mg-24 mg, 3 mg-15 mg, 3 mg-6 mg, 6 mg-600 mg, 6 mg-300 mg, 6 mg-150 mg, 6 mg-120 mg, 6 mg-96 mg, 6 mg-48 mg, 6 mg-24 mg, 6 mg-15 mg, 15 mg-600 mg, 15 mg-300 mg, 15 mg-150 mg, 15 mg-120 mg, 15 mg-96 mg, 15 mg-48 mg, 15 mg-24 mg, 24 mg-600 mg, 24 mg-300 mg, 24 mg-150 mg, 24 mg-120 mg, 24 mg-96 mg, 24 mg-48 mg, 48 mg-600 mg, 48 mg-300 mg, 48 mg-150 mg, 48 mg-120 mg, 48 mg-96 mg, 96 mg-600 mg, 96 mg-300 mg, 96 mg-150 mg, 96 mg-120 mg, 120 mg-600 mg, 120 mg-300 mg, 120 mg-150 mg, 150 mg-600 mg, 150 mg-300 mg or 300 mg-600 mg.

The second aspect of the present invention relates to a polypeptide for preventing and/or treating neuroblastoma, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

In some embodiments, the medicament is in a form of a white or off-white loose cake or powder. Further, the medicament should be diluted with 5% glucose solution (e.g., glucose injection), not with sodium chloride injection.

In some embodiments, wherein the medicament can be administered by intravenous injection.

In some embodiments, wherein the neuroblastoma is neuroblastoma in situ or metastatic neuroblastoma. Further, the neuroblastoma is liver metastases from neuroblastoma, lung metastases from neuroblastoma, brain metastases from neuroblastoma, bone metastases from neuroblastoma, lymphatic metastases from neuroblastoma, and myeloid metastases from neuroblastoma.

In some embodiments, the medicament is in a form of unit dosage. Further, the unit dosage comprises the following amounts of the polypeptide: about 0.6 mg, about 3 mg, about 6 mg, about 15 mg, about 24 mg, about 48 mg, about 96 mg, about 120 mg, about 150 mg, about 300 mg, about 600 mg, or a range between foregoing amounts of the polypeptide, for example, 0.6 mg-600 mg, 0.6 mg-300 mg, 0.6 mg-150 mg, 0.6 mg-120 mg, 0.6 mg-96 mg, 0.6 mg-48 mg, 0.6 mg-24 mg, 0.6 mg-15 mg, 0.6 mg-6 mg, 0.6 mg-3 mg, 3 mg-600 mg, 3 mg-300 mg, 3 mg-150 mg, 3 mg-120 mg, 3 mg-96 mg, 3 mg-48 mg, 3 mg-24 mg, 3 mg-15 mg, 3 mg-6 mg, 6 mg-600 mg, 6 mg-300 mg, 6 mg-150 mg, 6 mg-120 mg, 6 mg-96 mg, 6 mg-48 mg, 6 mg-24 mg, 6 mg-15 mg, 15 mg-600 mg, 15 mg-300 mg, 15 mg-150 mg, 15 mg-120 mg, 15 mg-96 mg, 15 mg-48 mg, 15 mg-24 mg, 24 mg-600 mg, 24 mg-300 mg, 24 mg-150 mg, 24 mg-120 mg, 24 mg-96 mg, 24 mg-48 mg, 48 mg-600 mg, 48 mg-300 mg, 48 mg-150 mg, 48 mg-120 mg, 48 mg-96 mg, 96 mg-600 mg, 96 mg-300 mg, 96 mg-150 mg, 96 mg-120 mg, 120 mg-600 mg, 120 mg-300 mg, 120 mg-150 mg, 150 mg-600 mg, 150 mg-300 mg or 300 mg-600 mg.

The third aspect of the present invention relates to a method for preventing and/or treating neuroblastoma, wherein the method comprises administering to a subject in need thereof a therapeutically effective amount of a polypeptide, the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1).

In some embodiments, wherein the medicament is in a form of a white or off-white loose cake or powder. Further, the medicament should be diluted with 5% glucose solution (e.g., glucose injection), not with sodium chloride injection.

In some embodiments, wherein the medicament can be administered by intravenous injection.

In some embodiments, wherein the neuroblastoma is neuroblastoma in situ or metastatic neuroblastoma. Further, the neuroblastoma is liver metastases from neuroblastoma, lung metastases from neuroblastoma, brain metastases from neuroblastoma, bone metastases from neuroblastoma, lymphatic metastases from neuroblastoma, and myeloid metastases from neuroblastoma.

In some embodiments, wherein the subject is a human patient. Further, the age of the human patient is 1 month to 18 years old. Further, the age of the human patient is 1 month to 14 years, 1 month to 12 years, 1 month to 10 years, 1 month to 8 years, 1 month to 6 years, 1 month to 3 years or 1 month to 1 year old.

In some embodiments, the daily administration amount of the polypeptide is about 0.01 mg/kg, about 0.05 mg/kg, about 0.1 mg/kg, about 0.25 mg/kg, about 0.4 mg/kg, about 0.8 mg/kg, about 1.6 mg/kg, about 2 mg/kg, about 2.5 mg/kg, about 5 mg/kg, about 10 mg/kg, or a range between foregoing amounts of the polypeptide, for example, 0.01 mg/kg-10 mg/kg, 0.01 mg/kg-5 mg/kg, 0.01 mg/kg-2.5 mg/kg, 0.01 mg/kg-2 mg/kg, 0.01 mg/kg-1.6 mg/kg, 0.01 mg/kg-0.8 mg/kg, 0.01 mg/kg-0.4 mg/kg, 0.01 mg/kg-0.25 mg/kg, 0.01 mg/kg-0.1 mg/kg, 0.01 mg/kg-0.05 mg/kg, 0.05 mg/kg-10 mg/kg, 0.05 mg/kg-5 mg/kg, 0.05 mg/kg-2.5 mg/kg, 0.05 mg/kg-2 mg/kg, 0.05 mg/kg-1.6 mg/kg, 0.05 mg/kg-0.8 mg/kg, 0.05 mg/kg-0.4 mg/kg, 0.05 mg/kg-0.25 mg/kg, 0.05 mg/kg-0.1 mg/kg, 0.1 mg/kg-10 mg/kg, 0.1 mg/kg-5 mg/kg, 0.1 mg/kg-2.5 mg/kg, 0.1 mg/kg-2 mg/kg, 0.1 mg/kg-1.6 mg/kg, 0.1 mg/kg-0.8 mg/kg, 0.1 mg/kg-0.4 mg/kg, 0.1 mg/kg-0.25 mg/kg, 0.25 mg/kg-10 mg/kg, 0.25 mg/kg-5 mg/kg, 0.25 mg/kg-2.5 mg/kg, 0.25 mg/kg-2 mg/kg, 0.25 mg/kg-1.6 mg/kg, 0.25 mg/kg-0.8 mg/kg, 0.25 mg/kg-0.4 mg/kg, 0.4 mg/kg-10 mg/kg, 0.4 mg/kg-5 mg/kg, 0.4 mg/kg-2.5 mg/kg, 0.4 mg/kg-2 mg/kg, 0.4 mg/kg-1.6 mg/kg, 0.4 mg/kg-0.8 mg/kg, 0.8 mg/kg-10 mg/kg, 0.8 mg/kg-5 mg/kg, 0.8 mg/kg-2.5 mg/kg, 0.8 mg/kg-2 mg/kg, 0.8 mg/kg-1.6 mg/kg, 1.6 mg/kg-10 mg/kg, 1.6 mg/kg-5 mg/kg, 1.6 mg/kg-2.5 mg/kg, 1.6 mg/kg-2 mg/kg, 2 mg/kg-10 mg/kg, 2 mg/kg-5 mg/kg, 2 mg/kg-2.5 mg/kg, 2.5 mg/kg-10 mg/kg, 2.5 mg/kg-5 mg/kg or 5 mg/kg-10 mg/kg.

In some embodiments, wherein the polypeptide is administered once a day.

In some embodiments, where the polypeptide is administered for 1 week, 2 weeks, or 3 weeks as needed.

The third aspect of the present invention relates to a composition comprising a polypeptide and an adjuvant, wherein the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1), the composition is used for preventing and/or treating of neuroblastoma.

In some embodiments, wherein the medicament is in a form of a white or off-white loose cake or powder. Further, the medicament should be diluted with 5% glucose solution (e.g., glucose injection), not with sodium chloride injection.

In some embodiments, wherein the adjuvant is mannitol.

In some embodiments, wherein the medicament can be administered by intravenous injection.

In some embodiments, wherein the neuroblastoma is neuroblastoma in situ or metastatic neuroblastoma. Further, the neuroblastoma is liver metastases from neuroblastoma, lung metastases from neuroblastoma, brain metastases from neuroblastoma, bone metastases from neuroblastoma, lymphatic metastases from neuroblastoma, and myeloid metastases from neuroblastoma.

In some embodiments, the daily administration amount of the polypeptide is about 0.01 mg/kg, about 0.05 mg/kg, about 0.1 mg/kg, about 0.25 mg/kg, about 0.4 mg/kg, about 0.8 mg/kg, about 1.6 mg/kg, about 2 mg/kg, about 2.5 mg/kg, about 5 mg/kg, about 10 mg/kg, or a range between foregoing amounts of the polypeptide, for example, 0.01 mg/kg-10 mg/kg, 0.01 mg/kg-5 mg/kg, 0.01 mg/kg-2.5 mg/kg, 0.01 mg/kg-2 mg/kg, 0.01 mg/kg-1.6 mg/kg, 0.01 mg/kg-0.8 mg/kg, 0.01 mg/kg-0.4 mg/kg, 0.01 mg/kg-0.25 mg/kg, 0.01 mg/kg-0.1 mg/kg, 0.01 mg/kg-0.05 mg/kg, 0.05 mg/kg-10 mg/kg, 0.05 mg/kg-5 mg/kg, 0.05 mg/kg-2.5 mg/kg, 0.05 mg/kg-2 mg/kg, 0.05 mg/kg-1.6 mg/kg, 0.05 mg/kg-0.8 mg/kg, 0.05 mg/kg-0.4 mg/kg, 0.05 mg/kg-0.25 mg/kg, 0.05 mg/kg-0.1 mg/kg, 0.1 mg/kg-10 mg/kg, 0.1 mg/kg-5 mg/kg, 0.1 mg/kg-2.5 mg/kg, 0.1 mg/kg-2 mg/kg, 0.1 mg/kg-1.6 mg/kg, 0.1 mg/kg-0.8 mg/kg, 0.1 mg/kg-0.4 mg/kg, 0.1 mg/kg-0.25 mg/kg, 0.25 mg/kg-10 mg/kg, 0.25 mg/kg-5 mg/kg, 0.25 mg/kg-2.5 mg/kg, 0.25 mg/kg-2 mg/kg, 0.25 mg/kg-1.6 mg/kg, 0.25 mg/kg-0.8 mg/kg, 0.25 mg/kg-0.4 mg/kg, 0.4 mg/kg-10 mg/kg, 0.4 mg/kg-5 mg/kg, 0.4 mg/kg-2.5 mg/kg, 0.4 mg/kg-2 mg/kg, 0.4 mg/kg-1.6 mg/kg, 0.4 mg/kg-0.8 mg/kg, 0.8 mg/kg-10 mg/kg, 0.8 mg/kg-5 mg/kg, 0.8 mg/kg-2.5 mg/kg, 0.8 mg/kg-2 mg/kg, 0.8 mg/kg-1.6 mg/kg, 1.6 mg/kg-10 mg/kg, 1.6 mg/kg-5 mg/kg, 1.6 mg/kg-2.5 mg/kg, 1.6 mg/kg-2 mg/kg, 2 mg/kg-10 mg/kg, 2 mg/kg-5 mg/kg, 2 mg/kg-2.5 mg/kg, 2.5 mg/kg-10 mg/kg, 2.5 mg/kg-5 mg/kg or 5 mg/kg-10 mg/kg.

### DETAILED DESCRIPTION OF THE PREFERRED EMBODIMENT

The present invention is further illustrated by the following examples, however, it should be understood that the following examples are only used for more detailed description, and should not be construed as being used to limit the present invention in any form.

### Example 1 Effect of HYD-PEP06 on Human Renal Metastases from Neuroblastoma in Athymic BALB/c/Nu/Nu Nude Mice

### 1. Experimental Materials and Experimental Methods

### (1) Experimental Materials

### i) Experimental Reagents

The amino acid sequence of HYD-PEP06 is shown in SEQ ID NO.1 (HARBIN YIDA PHARMACEUTICAL CO., LTD).

Sodium chloride injection (Shijiazhuang No. 4 Pharmaceutical).

5% glucose injection (Sichuan Kelun Pharmaceutical Co., Ltd.)

Ketamine (Fujian Gutian Pharmaceutical Co., Ltd.)

Xylazine (Sigma-Aldrich, USA)

Firefly luciferase substrate Luciferin (PerkinElmer Inc.)

Fetal bovine serum (BeiJing YuanHeng ShengMa Biology Technology Research Institute)

DMEM (Gibco, USA)

Puromycine (Invivogen, USA)

Penicillin/streptomycin stock solution (Chinese Academy of Medical Sciences & Peking Union Medical College Institute of Biomedical Engineering)

Trypsin (Chinese Academy of Medical Sciences & Peking Union Medical College Institute of Biomedical Engineering)

### ii) Tumor Cell Lines

Human-derived neuroblastoma SH-SY5Y cell line, gifted by Prof. Aiping Wang, Institute of Materia Medica, Chinese Academy of Medical Sciences & Peking Union Medical College.

iii) Experimental Animals and Feeding Conditions

Adult athymic female nude mice (BALB/c/Nu/Nu, 18-22 g), purchased from Beijing Vital River Laboratory Animal Technology Co., Ltd. Mice were randomized over groups of five and housed in pathogen-free IVC cages around 25° C. with 40-70% humidity and 12 h light/dark cycle. The corncob bedding was sterilized by ⁶⁰Co radiation with a particle size of 4-6 mm. Nude mice were fed with sterilized chow and water ad libitum.

### iv) Experimental Instruments and Equipment

PekinElmer IVIS Spectrum CT imaging system, assorted anesthesia machine and Living image 4.5.1 software.

### (2) Establishment of Experimental Models

### i) Establishment of SH-SY5Y Cells Stably Expressing Firefly Luciferase (Luc2)

SH-SY5Y cells were infected with pCDH-luc2-GFP lentiviruses. The culture medium was replaced with DMEM supplement with 10% FBS (fetal bovine serum) after 24 h infection, followed by selection of 1 μg ml⁻¹ puromycin. After 7 days of culture, the expression of GFP in cells was observed under a fluorescence microscope, and the bioluminescene of cell was detected with a PekinElmer IVIS Spectrum CT imaging system.

### ii) Cell Culture

The cells were cultured in DMEM containing penicillin-streptomycin supplement with 10% FBS at 37° C. in 5% CO₂, and the medium was replaced every 1-2 days. The cells were digested with 0.25% trypsin, followed by centrifuging at 1000 r/min for 5 minutes, then the supernatant was removed, and fresh medium was added for further culture.

iii) Establishment of subcutaneous xenograft model of human neuroblastoma SH-SY5Y-luc2-GFP cells in logarithmic growth phase were harvested after trypsin treatment, and then washed with PBS and resuspended to a final cell concentration of 1×10⁷ cells/ml. Cell suspension of 2×10⁶ in 0.2 mL was injected (s.c.) into the back of nude mice to establish the traditional tumour xenograft nude mouse model.

### iv) Establishment of Renal Metastases Subcutaneous Xenograft Model of Human Neuroblastoma

When the SH-SY5Y-luc2-GFP subcutaneous xenografts grew to about 1-2 cm in diameter, the tumor was removed under pathogen-free conditions, and the tumor was cut into pieces of about 1.0 mm³ for later use. The nude mice were anesthetized with anesthetic (10 ml/kg), then fixed on the operating table. Next, the skin on the back was disinfected and covered with surgical drape, and the surgical drape was cut for an incision with about 1 cm on the right side of the back kidney to expose the right kidney. The prepared tumor pieces were placed into a special inoculation cannula needle, and the tumor pieces were implanted into the kidney with the cannula, and the bleeding area of the wound was treated with sterile gauze to stop bleeding. Then the kidney was put back into the nude mouse, and the dorsal muscle and skin were sutured sequentially with surgical sutures.

v) Bioluminescence Detection of Renal Metastases from Human Neuroblastoma in Athymic BALB/c/Nu/Nu Nude Mice

IVIS Spectrum CT imaging system was used for detection at each detection time point. The procedure was as follows: the subject nude mice were pre-anesthetized with 2% isoflurane in the anesthesia machine; the subject nude mice were placed in IVIS Spectrum CT room and maintained in anesthesia with 0.5% isoflurane; the bioluminescence imaging mode was selected to detect the in vivo luminescence signals; the signals were quantified using the Living image 4.5.1 software assorted with IVIS Spectrum CT imaging system.

### (3) Experimental Grouping and Treatment Regimen

Three days after the surgical modeling, the tumor-bearing mice were imaged with IVIS Spectrum CT in vivo, and the tumor-bearing mice were randomized over groups of 10 mice according to the luminescence intensity in the software analysis results. The tumor-bearing was divided into model control group, HYD-PEP06 5 mg/kg group, HYD-PEP06 10 mg/kg group and HYD-PEP06 20 mg/kg group. The tested medicament HYD-PEP06 was administered intravenously for 15 days, once a day.

Animals were observed daily and weighed every other day. Luminescence was detected with four times of in vivo imaging at day 1 (i.e., 3 days after surgical modeling), day 6, day 12 and day 15 from the experimental grouping. The time of in vivo imaging for luminescence detection was adjusted according to the survival and physical condition of the animals. At the end of the experiment (day 16) the animals were executed by neck dislocation, the kidney tissue and tumor masses was isolated for weighing and photographing, and the tumor masses were preserved in 4% formaldehyde solution for routine pathological testing.

### (4) Data Processing

Data were shown as mean±SEM; suppression rate of tumor growth in luminescence intensity=(luminescence intensity of model control group−luminescence intensity of administration group)/luminescence intensity of model control group×100%; tumor inhibition rate of the kidney in affected side=(weight of the kidney in affected side of the model control group−weight of the kidney in affected side of the administration group)/weight of the kidney in affected side of the model control group×100%; GraphPad Prism 5 software was used for statistical testing. The statistical analysis between groups was one-way ANOVA and Tukey's test for pairwise comparison, and the statistical analysis between groups at different time points was two-way ANOVA and Bonferroni test for pairwise comparison.

### 2. Experimental Results

In this study, renal metastases from human-derived neuroblastoma were used to evaluate the pharmacodynamic characteristics of HYD-PEP06, and tumor multiplication rates and medicament treatment effects was calculated according to tumor bioluminescence intensity. Compared with the tumor luminescence intensity at the time of experimental grouping (day 1), the tumor luminescence intensity of the model control nude mice increased 493.7-fold after 15 days (day 15), while the tumor luminescence intensity of the administration groups of 5 mg/kg, 10 mg/kg and 20 mg/kg of the tested medicament HYD-PEP06 was significantly lower than that of the model control group, which increased only 224.2-fold, 96.9-fold and 51.6-fold, respectively, showing a significant dose-effect relationship (see Table 3). At the end of the experiment, the tumor multiplication rate of each administration group of HYD-PEP06 was significantly reduced compared with the model control group (P<0.001). The tumor suppression rate was calculated based on the bioluminescence intensity of each group of tumor-bearing nude mice, and the tumor suppression rates of the 5 mg/kg, 10 mg/kg and 20 mg/kg administration groups of the tested medicament HYD-PEP06 were 58.4%, 82.5% and 91.1%, respectively, showing a significant dose-effect relationship (see Tables 1 and 2).

At the end of the experiment, the tumor suppressive effect of the medicament was analyzed by the aid of renal weight and renal weight index. The mean kidney weight in affected side of the tumor-bearing nude mice in the model control group was 2.516 g, while the mean kidney weight in affected side of the tumor-bearing nude mice in the administration groups of 5 mg/kg, 10 mg/kg and 20 mg/kg of HYD-PEP06 were 1.537 g, 1.130 g and 0.664 g, respectively, with significant differences between each treatment group and the model control group (P<0.05, P<0.001, P<0.001). The mean renal weight/body mass index in affected side of the model control group of tumor-bearing nude mice was 12.25%, while the administration groups of 5 mg/kg, 10 mg/kg and 20 mg/kg of the tested medicament HYD-PEP06 had such index of 7.35%, 5.42% and 3.12%, respectively, which was significantly lower than that of the model control group (P<0.05, P<0.001, P<0.001, respectively) and showed a significant dose-effect relationship (see Table 1). The mean left and right renal weight/body mass index of normal non tumor-bearing athymic BALB/c/Nu/Nu nude mice (10 mice) was 0.78%. Although the administration groups failed to restore the renal weight and renal weight/body mass index to normal value in the tumor-bearing nude mice, the tested medicament had shown a significant reduction effect on beared tumor and a significant delay effect on disease progression in the tumor-bearing nude mice. No significant differences were observed in kidney weight and kidney weight/body mass index (see Table 4) and body weight (see Table 5) in the normal side (left side) between the tumor-bearing nude mice groups and the model control group. The above results indicated that HYD-PEP06 did not show significant toxic side effects at the effective dose.

### SUMMARY

In conclusion, HYD-PEP06 was able to significantly inhibit tumor growth in nude mouse model with SH-SY5Y, a renal metastases from human-derived neuroblastoma, in a significant dose-effect relationship, and did not show significant toxic side effects at the effective dose. Therefore, HYD-PEP06 can be used for preventing and treating tumors associated with neuroblastoma.

TABLE 1
Effects of HYD-PEP06 on renal metastases from human neuroblastoma in athymic
BALB/c/Nu/Nu nude mice

| | | | | | | | | | Kidney in affected side | | |
|---|---|---|---|---|---|---|---|---|---|---|---|
| | | | | | | | | | | Sup- | |
| | | | | | | | Bioluminescence | | | pression | Kidney |
| | | | | | | | BLI | | | rate of | weight/ |
| | Mode and | Number | | | | | Intensity | | Kidney | renal | body mass |
| | days of | of | Body weight (g, | | Relative body weight | | (×10⁸ p/s, | Tumor | weight (g, | weight in | index (%, |
| | admini- | animals | mean ± SEM) | | (%, mean ± SEM) | | mean ± | suppression | Mean ± | affected | mean ± |
| Group | stration | Start/end | Start | End | Start | End | SEM | rate (%) | SEM) | side (%) | SEM) |
| Model | — | 10/10 | 20.7 ± 0.4 | 20.7 ± 0.4 | 100.0 ± 0.0 | 100.0 ± 1.5 | 181.3 ± | — | 2.516 ± | — | 12.25 ± |
| control | | | | | | | 43.6 | | 0.331 | | 1.63 |
| group | | | | | | | | | | | |
| HYD- | i.v. × 15 | 10/10 | 20.8 ± 0.6 | 21.0 ± 0.4 | 100.0 ± 0.0 | 101.6 ± 1.9 | 75.4 ± | 58.4 | 1.537 ± | 38.9 | 7.35 ± |
| PEP06 | | | | | | | 14.9* | | 0.183* | | 0.92* |
| 5 mg/kg | | | | | | | | | | | |
| group | | | | | | | | | | | |
| HYD- | i.v. × 15 | 10/10 | 20.2 ± 0.4 | 21.0 ± 0.4 | 100.0 ± 0.0 | 104.0 ± 2.2 | 31.7 ± | 82.5 | 1.130 ± | 55.1 | 5.42 ± |
| PEP06 | | | | | | | 5.6*** | | 0.136*** | | 0.67*** |
| 10 mg/kg | | | | | | | | | | | |
| group | | | | | | | | | | | |
| HYD- | i.v. × 15 | 10/10 | 20.3 ± 0.4 | 21.3 ± 0.6 | 100.0 ± 0.0 | 104.9 ± 3.3 | 16.2 ± | 91.1 | 0.664 ± | 73.6 | 3.12 ± |
| PEP06 | | | | | | | 3.6 | | 0.073***^{,&} | | 0.34***^{,&} |
| 20 mg/kg | | | | | | | | | | | |
| group | | | | | | | | | | | |

Compared with the model control group,
*P < 0.05,
**P < 0.01,
***P < 0.001; compared with the HYD-PEP06 5 mg/kg group,
^{&}P < 0.05 (One-way ANOVA and Tukey's test for two-way comparison)

TABLE 2
Absolute values of bioluminescence signal intensity of tumor-bearing nude mice in each
group over time

| | | | | | Tumor inhibition | | |
|---|---|---|---|---|---|---|---|
| | | | | | rate (%) | | |
| | BLI luminescence intensity (×10⁷ p/s, mean ± SEM) | | | | Day | Day | Day |
| Group | Day 1 | Day 6 | Day 12 | Day 15 | 6 | 12 | 15 |
| Model | 4.29 ± 0.80 | 54.64 ± 13.72 | 754.44 ± 151.62 | 1812.80 ± 436.25 | | | |
| control | | | | | | | |
| group | | | | | | | |
| HYD-PEP06 | 4.27 ± 0.76 | 24.66 ± 2.54 | 473.17 ± 92.47 | 753.70 ± 149.24*** | 54.9 | 37.3 | 58.4 |
| 5 mg/kg | | | | | | | |
| group | | | | | | | |
| HYD-PEP06 | 4.23 ± 0.73 | 13.31 ± 3.46 | 178.66 ± 47.48** | 316.78 ± 55.71*** | 75.6 | 76.3 | 82.5 |
| 10 mg/kg | | | | | | | |
| group | | | | | | | |
| HYD-PEP06 | 4.19 ± 0.67 | 10.40 ± 3.01 | 105.73 ± 20.43*** | 161.64 ± 35.65*** | 81.0 | 86.0 | 91.1 |
| 20 mg/kg | | | | | | | |
| group | | | | | | | |

Compared with the model control group,
*P < 0.05,
**P < 0.01,
***P < 0.001(Two-way ANOVA and Bonferroni test for two-way comparison)

TABLE 3
Statistics of the fold increase of bioluminescence signal intensity
of tumor-bearing nude mice in each group over time

| | Relative bioluminescence intensity (mean ± SEM) | | | |
|---|---|---|---|---|
| Group | Day 1 | Day 6 | Day 12 | Day 15 |
| Model control | 1.00 ± 0.00 | 16.4 ± 3.6 | 232.2 ± 59.5 | 493.7 ± 96.7 |
| group | | | | |
| HYD-PEP06 | 1.00 ± 0.00 | 7.6 ± 1.8 | 142.5 ± 33.4 | 224.2 ± |
| 5 mg/kg group | | | | 46.9*** |
| HYD-PEP06 | 1.00 ± 0.00 | 4.4 ± 1.3 | 71.4 ± 29.9** | 96.9 ± |
| 10 mg/kg group | | | | 29.6*** |
| HYD-PEP06 | 1.00 ± 0.00 | 2.8 ± 0.7 | 32.4 ± 8.7*** | 51.6 ± |
| 20 mg/kg group | | | | 15.1*** |

Compared with the model control group,
*P < 0.05,
**P < 0.01,
***P < 0.001(Two-way ANOVA and Bonferroni test for two-way comparison)

TABLE 4
Statistical of kidney weight and kidney index on normal
side (left side) of tumor-bearing nude mice in each group

| | Kidney weight | Renal weight/body |
|---|---|---|
| | on normal side | mass index on normal side |
| Group | (g, mean ± SEM) | (%, mean ± SEM) |
| Model control group | 0.171 ± 0.004 | 0.83 ± 0.02 |
| HYD-PEP06 | 0.178 ± 0.003 | 0.85 ± 0.01 |
| 5 mg/kg group | | |
| HYD-PEP06 | 0.178 ± 0.006 | 0.85 ± 0.02 |
| 10 mg/kg group | | |
| HYD-PEP06 | 0.182 ± 0.005 | 0.86 ± 0.01 |
| 20 mg/kg group | | |
| Normal tumor ± | 0.173 ± 0.003 | 0.79 ± 0.01 |
| free mice | | |

TABLE 5
Statistical of changes in body weight of tumor-bearing
nude mice in each group over time

| | Body weight of tumor-bearing | | | | | |
|---|---|---|---|---|---|---|
| | nude mice (g, mean ± SEM) | | | | | |
| Group | Day 1 | Day 4 | Day 7 | Day 10 | Day 13 | Day 16 |
| Model | 20.7 ± | 22.1 ± | 21.4 ± | 21.0 ± | 21.5 ± | 20.7 ± |
| control | 0.4 | 0.5 | 0.5 | 0.4 | 0.3 | 0.4 |
| group | | | | | | |
| HYD- | 20.8 ± | 21.8 ± | 21.2 ± | 20.9 ± | 21.1 ± | 21.0 ± |
| PEP06 | 0.6 | 0.6 | 0.6 | 0.5 | 0.4 | 0.4 |
| 5 mg/kg | | | | | | |
| group | | | | | | |
| HYD- | 20.2 ± | 21.9 ± | 21.1 ± | 21.2 ± | 21.3 ± | 21.0 ± |
| PEP06 | 0.4 | 0.3 | 0.3 | 0.3 | 0.2 | 0.4 |
| 10 mg/kg | | | | | | |
| group | | | | | | |
| HYD- | 20.3 ± | 21.6 ± | 20.9 ± | 20.9 ± | 21.3 ± | 21.3 ± |
| PEP06 | 0.4 | 0.4 | 0.5 | 0.5 | 0.6 | 0.6 |
| 20 mg/kg | | | | | | |
| group | | | | | | |

What is claimed is:

1. A method for preventing and/or treating neuroblastoma, wherein the method comprises administering to a subject in need thereof a therapeutically effective amount of a polypeptide or a pharmaceu, the polypeptide has an amino acid sequence of Arg-Gly-Asp-Arg-Gly-Asp-Met-His-Ser-His-Arg-Asp-Phe-Gln-Pro-Val-Leu-His-Leu-Val-Ala-Leu-Asn-Ser-Pro-Leu-Ser-Gly-Gly-Met (SEQ ID NO: 1);
wherein the neuroblastoma is metastatic neuroblastoma.

2. The method of claim 1, wherein the polypeptide has a daily administration amount from 0.1 mg/kg to 10 mg/kg.
